(19) United States
(12) Patent Application Publication
(10) Pub. No.: US 2016/0143331 A1
(43) Pub. Date: May 26, 2016

(54) LIQUID FOOD COMPOSITION

(71) Applicant: KANEKA CORPORATION, Osaka-shi (JP)

(72) Inventors: Hiroaki INOUE, Takasago-shi (JP); Masao SATO, Takasago-shi (JP); Tadashi MOROSHIMA, Takasago-shi (JP); Ken UEKITA, Takasago-shi (JP); Shinichi YOKOTA, Takasago-shi (JP); Yui KAWASHIMA, Shinagawa (JP); Tadaki HASHIMOTO, Shinagawa-ku (JP); Kazuya HAMADA, Takasago-shi (JP)

(73) Assignee: KANEKA CORPORATION, Osaka-shi, Osaka (JP)

(21) Appl. No.: 14/897,893

(22) PCT Filed: Jun. 16, 2014
(86) PCT No.: PCT/JP2014/065842

§ 371 Date: Dec. 11, 2015

(30) Foreign Application Priority Data
Jun. 17, 2013 (JP) 2013-126769
Oct. 16, 2013 (JP) 2013-215371

(51) Int. Cl. A61K 38/16; A61K 31/716; A61K 31/734; A61K 36/899; A61K 36/48; A61K 9/00; A23L 1/212

(52) U.S. Cl. CPC A23L 1/296; A61K 38/168; A61K 31/734; A61K 31/716; A61K 36/899; A61K 36/48; A61K 9/0053; A23L 1/304; A23L 1/0524; A23L 1/0532; A23L 1/3055; A23V 2002/00

(57) ABSTRACT

A liquid food composition that can be conveniently ingested or given by tube feeding, and can prevent gastroesophageal reflux disease, vomiting, esophagitis, pneumonia, asphyxiation, diarrhea, etc., and can increase feeling of fullness through improvement in the solidification ratio of the composition under the endogastric condition is provided. Since the liquid food composition of the present invention shows superior solidification ratio under the endogastric acidic condition, it can more effectively reduce the risks for developing gastroesophageal reflux disease, vomiting, esophagitis, pneumonia, asphyxiation, diarrhea, etc., and it does not require labor for separately adding a gelling agent etc. at the time of ingestion, and since it is liquid, it can be easily ingested by tube feeding.

### TECHNICAL FIELD

The present invention relates to a liquid food composition that is used when, for example, an elderly person, person with a disease, preoperative or postoperative patient, or healthy subject takes nutrition, and relates to a nutritious food, especially a liquid food, as well as a food for enteral feeding, a nutrient preparation for enteral feeding, etc. usable by tube feeding.

### BACKGROUND ART

In recent years, nutritious foods produced in consideration of nutritional balance are used not only as conveniently ingestible takeout foods and diet foods, but also as liquid foods for intake of nutrition by those who suffer from difficulty in oral ingestion of foods due to advanced age, sickness, wounds or disability. However, at the time of intake of liquid foods, liquid foods may flow backward into the esophagus from the stomach (gastroesophageal reflux disease), which may cause even die by inducing vomiting, esophagitis, pneumonia, asphyxiation, and so forth. Moreover, liquid foods are in the form of liquid showing low viscosity, and therefore they also pose a problem that when liquid foods promptly flow into the intestines, they easily cause osmotic diarrhea.

As means for solving such problems, there have been disclosed a method of giving a preliminarily gelled nutritious food (Patent document 1), a method of adding a gelling agent to a nutritious food at the time of ingestion of the food to reduce fluidity of the nutritious food in the stomach (Patent document 2), and a composition containing a polysaccharide, a mineral, and a gas-forming component, which is gelled under the endogastric condition (Patent document 3).

Further, for the purpose of improving gel strength in various foods, there has been proposed a method of concentrating a fraction of 7S globulin and/or 15S globulin, which are constitutional ingredients of vegetable proteins, and adding the concentrated fraction to a food (Patent documents 4 and 5).

### PRIOR ART REFERENCES

### Patent Documents

Patent document 1: Japanese Patent Unexamined Publication (KOKAI) No. 2006-182767
Patent document 2: Japanese Patent Unexamined Publication (KOKAI) No. 2007-176848
Patent document 3: Japanese Patent Unexamined Publication (KOHYO) No. 2009-524575
Patent document 4: WO08/041572
Patent document 5: Japanese Patent Unexamined Publication (KOHYO) No. 2008-513490

### SUMMARY OF THE INVENTION

### Object to be Achieved by the Invention

However, for example, the method described in Patent document 1 has a characteristic that the reduced fluidity of the nutritious food makes it difficult to pass the nutritious food through a tube at the time of tube feeding. Therefore, it has problems, for example, ingestion of nutritious foods takes a long time, which results in burden on persons who ingest them, or causes development and aggravation of bedsore due to continuance of seating position. The method described in Patent document 2 requires separate addition of a gelling agent to nutritious foods, and it makes the operation more complicated. Therefore, at the time of using a nutritious food, the preparation of the nutritious food requires labor and time, and risks are also expected in view of sanitation concerning, for example, contamination with bacteria during the operation.

The composition described in Patent document 3 can be expected to show an effect of ameliorating the aforementioned problems to a certain extent. However, since the composition of Patent document 3 cannot provide sufficient gelation of the composition under the stomach conditions, it cannot sufficiently reduce the risks for developing gastroesophageal reflux disease, vomiting, esophagitis, pneumonia, asphyxiation, diarrhea, and so forth. Thus, the aforementioned problems are not solved yet.

In order to solve the problem of the composition of Patent document 3, the inventors of the present invention examined, for example, addition of the vegetable proteins described in Patent documents 4 and 5 to liquid food compositions containing water-soluble dietary fibers, minerals, and so forth. However, the problem of developing gastroesophageal reflux disease, vomiting, esophagitis, pneumonia, asphyxiation, diarrhea, etc. could not be ameliorated by increasing the gel strength of the gel-like product formed under the endogastric condition. That is, they found that the aforementioned problems were caused by presence of unsolidified matter, which did not solidify and remain in the stomach, and it was important to reduce such unsolidified matter, i.e., it was important to improve efficiency of solidification of a composition under the endogastric condition.

In view of the aforementioned problems etc., an object of the present invention is to provide a liquid food composition that can be conveniently ingested or given by tube feeding, and can prevent gastroesophageal reflux disease, vomiting, esophagitis, pneumonia, asphyxiation, diarrhea, etc. and can increase feeling of fullness through improvement in the solidification ratio of the composition under the endogastric condition.

### Means for Achieving the Object

The inventors of the present invention conducted various researches in order to achieve the aforementioned object, and as a result, found that, by blending a vegetable protein having a reduced content of molecules constituting proteins such as globulins, water-soluble dietary fibers, a divalent metal salt, etc., a composition showing an improved solidification ratio under the endogastric condition could be provided. In particular, they found that the intended effect could be fully expected for such a composition as mentioned above showing a solidification ratio higher than 56%, and accomplished the present invention.

The present invention is thus embodied as follows.

(1) A liquid food composition that shows fluidity at pH 5.5 to 10.0, and thickens and/or solidifies at a pH lower than 5.5, wherein the composition contains one or more kinds of substances selected from the group consisting of alginic acid, a salt thereof, and pectin, a divalent metal salt, and a vegetable protein, and the vegetable protein shows a relative mobility (Rf value) larger than 0.6 at a cumulative value of pixel intensity frequency of 50% in SDS-PAGE densitometry analysis, or a liquid food composition that shows fluidity at pH 5.5 to 10.0, and thickens and/or solidifies at a pH lower than 5.5, wherein the composition contains one or more kinds of substances selected from the group consisting of alginic acid, a salt thereof, and pectin, a divalent metal salt, and a vegetable protein, and 7S globulin content of the vegetable protein is not lower than 0.01% by weight and lower than 21% by weight, and/or 11S globulin content of the vegetable protein is not lower than 0.01% by weight and lower than 41% by weight.
(2) The liquid food composition according to (1), wherein the 7S globulin content of the vegetable protein is not lower than 0.01% by weight and lower than 21% by weight, and the 11S globulin content of the vegetable protein is not lower than 0.01% by weight and lower than 41% by weight.
(3) The liquid food composition according to (2), wherein ratio of the 11S globulin and 7S globulin contents ([11S globulin]/[7S globulin], weight basis) is larger than 0.0005 and smaller than 4100.
(4) The liquid food composition according to any one of (1) to (3), wherein ratio of contents of the one or more kinds of substances selected from the group consisting of alginic acid, a salt thereof, and pectin, and the vegetable protein ([one or more kinds of substances selected from the group consisting of alginic acid, a salt thereof, and pectin]/[vegetable protein], weight basis) is not smaller than 0.05 and not larger than 4.
(5) The liquid food composition according to any one of (2) to (4), wherein ratio of contents of the one or more kinds of substances selected from the group consisting of alginic acid, a salt thereof, and pectin, and 7S globulin ([one or more kinds of substances selected from the group consisting of alginic acid, a salt thereof, and pectin]/[7S globulin], weight basis) is larger than 0.07 and not larger than 200000.
(6) The liquid food composition according to any one of (2) to (5), wherein ratio of contents of the one or more kinds of substances selected from the group consisting of alginic acid, a salt thereof, and pectin, and 11S globulin ([one or more kinds of substances selected from the group consisting of alginic acid, a salt thereof, and pectin]/[11S globulin], weight basis) is larger than 0.04 and not larger than 200000.
(7) The liquid food composition according to any one of (1) to (6), wherein the vegetable protein consists of soybean proteins.
(8) The liquid food composition according to any one of (2) to (7), wherein the 7S globulin is β-conglycinin.
(9) The liquid food composition according to any one of (2) to (8), wherein the 11S globulin is glycinin.
(10) The liquid food composition according to any one of (1) to (9), wherein the divalent metal salt is a calcium compound and/or a magnesium compound.
(11) The liquid food composition according to any one of (1) to (10), which is filled in a container connectable with a nasogastric catheter or a gastrostomy catheter.
(12) The liquid food composition according to any one of (1) to (11), which is used in a treatment of a disease or condition that requires liquid food or tube feeding.
(13) A method for improving thickening and/or solidification of a liquid food composition at a pH lower than 5.5, which comprises using, in a liquid food composition containing one or more kinds of substances selected from the group consisting of alginic acid, a salt thereof, and pectin, a divalent metal salt, and a vegetable protein, a vegetable protein that shows an Rf value larger than 0.6 as the vegetable protein.

### Effect of the Invention

Since the liquid food composition of the present invention shows superior solidification efficiency under the acidic condition in the stomach, it can more effectively reduce the risks for developing gastroesophageal reflux disease, aspiration pneumonia, diarrhea, etc. Further, it does not require labor for separately adding a gelling agent etc. at the time of ingestion, and since it is liquid, it can be easily ingested by tube feeding.

### BRIEF DESCRIPTION OF THE DRAWINGS

FIG. 1 shows the results of the densitometry analyses performed in Examples 1 and 2 of the present invention, and Comparative Example 1.

FIG. 2 shows the SDS-PAGE patterns of soybean protein raw materials obtained in Examples 3 and 4 of the present invention, and Comparative Example 2.

### MODES FOR CARRYING OUT THE INVENTION

Hereafter, the present invention will be explained in detail.

When pH of the liquid food composition of the present invention is in the neutral range, the physical properties of the composition as liquid are stably maintained. Further, when pH of the composition falls within the acidic region in the stomach after ingestion, the composition changes the form thereof from liquid to solid. That is, the form of the liquid food composition of the present invention is liquid at the time of production, distribution, preservation, ingestion thereof etc. Further, it has a property that when it is mixed with stomach juice after ingestion, the form thereof changes into solid.

In the present invention, the neutral condition is not particularly limited so long as the physical properties of the liquid food composition as liquid are not degraded. As for the lowest pH of the neutral condition, pH is preferably 5.5 or higher, more preferably 6.0 or higher, further preferably 6.5 or higher. Further, as for the highest pH of the neutral condition, pH is preferably 10.0 or lower, more preferably 9.5 or lower, further preferably 9.0 or lower, irrespective of how the lowest pH is defined. If pH is lower than 5.5, it is difficult to maintain the physical properties of the composition as liquid, and when pH is higher than 10.0, deterioration or decomposition of nutritional components contained in the composition are concerned, and therefore such a pH is not preferred.

In the present invention, the acidic condition is not particularly limited so long as the form of the liquid food composition changes into solid under the acidic condition. As for the highest pH of the acidic condition, pH is preferably lower than 5.5, more preferably 5.0 or lower, further preferably 4.5 or lower, particularly preferably 4.0 or lower. If pH is 5.5 or higher, change of the form of the composition into solid under the acidic condition may not be fully attained, and therefore such a pH is not preferred.

In the present invention, the terms or expressions “liquid”, “physical properties as liquid”, and “showing fluidity” mean that the food composition is in a state that it can be fed via a tube (for example, nasogastric tube, and gastrostomy tube). Although viscosity of the composition is not particularly limited so long as the convenience at the time of the feeding via a tube is not degraded, the viscosity is, for example, preferably 1000 cP or lower, more preferably 500 cP or lower, further preferably 300 cP or lower, particularly preferably 200 cP or lower. The values of viscosity indicated in this description are values measured at 25° C., unless otherwise indicated.

In the present invention, the terms “solidification”, “change of form into solid”, or “to thicken and/or solidify” mean change of the physical properties of the liquid food composition as liquid under the acidic condition, such as insolubilization, increase in viscosity, solation, and gelation of the liquid food composition, and these can be evaluated in terms of solidification ratio.

In the present invention, the solidification ratio is an index for indicating efficiency of the change of the form of the liquid food composition under the acidic condition. The liquid food composition showing a higher solidification ratio more efficiently changes into solid, and leaves a smaller volume of liquid portion (unsolidified portion).

According to the present invention, the solidification ratio is preferably higher than 56%, more preferably not lower than 65%, further preferably not lower than 70%, particularly preferably not lower than 80%. The values of the solidification ratio referred to in the present invention are values measured by the method described in the examples, the section of <Test for confirming solidification ratio under acidic condition> contained in this description.

The liquid food composition of the present invention contains one or more kinds of substances selected from the group consisting of alginic acid, a salt thereof, and pectin, or alginic acid and/or a salt thereof, and pectin. As the salt of alginic acid, sodium salt, potassium salt, and ammonium salt are preferably used, and among them, sodium alginate is especially preferably used from the viewpoint of favorable change of the form from liquid to solid. As pectin, high-methoxylated (HM) pectin and low-methoxylated (LM) pectin are preferably used, and among them, LM-pectin is especially preferably used, from the viewpoint of favorable change of the form from liquid to solid. Further, they can be used independently or as a combination of two or more kinds of them. As for the minimum amount of the one or more kinds of substances selected from the group consisting of alginic acid, a salt thereof, and pectin (when two or more kinds of substances are used, total amount of them) with respect to the liquid food composition, they are preferably used in an amount of 0.3% by weight or larger, more preferably 0.5% by weight or larger, further preferably 0.7% by weight or larger, particularly preferably 1.0% by weight or larger. If the amount is smaller than 0.3% by weight, solidification of the liquid food composition under the acidic condition may become insufficient, and therefore such an amount is not preferred. As for the maximum amount of the one or more kinds of substances selected from the group consisting of alginic acid, a salt thereof, and pectin, they are preferably used in an amount of 5.0% by weight or smaller, more preferably 2.5% by weight or smaller, further preferably 2.0% by weight or smaller, particularly preferably 1.5% by weight or smaller, irrespective of how the minimum amount is defined. If the amount is larger than 5.0% by weight, the physical properties of the liquid food composition may be degraded because of increase in viscosity, and therefore such an amount is not preferred.

The liquid food composition of the present invention contains a divalent metal salt. In the present invention, the divalent metal salt can be selected from a calcium compound and/or a magnesium compound. As the calcium compound, for example, calcium chloride, calcium gluconate, calcium acetate, calcium hydroxide, calcium sulfate, tricalcium phosphate, calcium carbonate, calcium citrate, calcium dihydrogenpyrophosphate, calcium monohydrogenphosphate, calcium stearate, and calcium silicate are preferably used, among them, a calcium compound poorly soluble under the neutral condition is more preferably used, and calcium carbonate, calcium dihydrogenpyrophosphate, and tricalcium phosphate are further preferably used. As the magnesium compound, magnesium chloride, magnesium acetate, magnesium hydroxide, magnesium sulfate, magnesium carbonate, magnesium oxide, magnesium stearate, trimagnesium phosphate, magnesium silicate, and so forth are preferably used, among them, a magnesium compound poorly soluble under the neutral condition is more preferably used, and magnesium carbonate and magnesium oxide are further preferably used. These calcium compounds and/or magnesium compounds may be used independently, or in a combination of two or more kinds of them.

The term “poorly-soluble” referred to in the present invention means a property of from “Sparingly soluble” to “Practically insoluble” according to the solubility standards described in Japanese Pharmacopoeia, General Notices. More precisely, it means that when water containing a solute is vigorously shaken for 30 seconds every 5 minutes at 20±5° C., volume of water required for dissolving 1 g or 1 ml of the solute within 30 minutes is 30 ml or larger. In the present invention, solubility of the poorly soluble calcium compound and/or poorly soluble magnesium compound in water at 20±5° C. is preferably 100 mg/100 ml or lower, more preferably 75 mg/100 ml or lower, further preferably 50 mg/100 ml or lower.

As the divalent metal salt, any of those mentioned above may be used, and combination thereof is not also particularly limited. However, in view of solubility in the neutral region and suitability for use in foods, a combination of calcium carbonate and magnesium carbonate is preferred. The content of the divalent metal salt in the liquid food composition is not particularly limited so long as it is used in an amount nutritionally sufficient for a person who ingests or gives the liquid food composition, but the minimum content of calcium is, for example, 2 μg/100 ml or higher, preferably 2 mg/100 ml or higher, more preferably 20 mg/100 ml or higher, further preferably 50 mg/100 ml or higher, still further preferably 100 mg/100 ml or higher, particularly preferably 150 mg/100 ml or higher. As for the maximum content of the divalent metal salt, it is, for example, 3500 mg/100 ml or lower, preferably 2400 mg/100 ml or lower, more preferably 1100 mg/100 ml or lower, further preferably 600 mg/100 ml or lower, particularly preferably 350 mg/100 ml or lower, irrespective of how the minimum content is defined. When calcium carbonate and magnesium carbonate are used in combination, the minimum content of calcium in the liquid food composition is, for example, 1 μg/100 ml or higher, preferably 1 mg/100 ml or higher, more preferably 10 mg/100 ml or higher, further preferably 30 g/100 ml or higher, still further preferably 50 mg/100 ml or higher, particularly preferably 75 mg/100 ml or higher, irrespective of the magnesium content. The maximum calcium content is, for example, 3000 mg/100 ml or lower, preferably 2000 mg/100 ml or lower, more preferably 1000 mg/100 ml or lower, further preferably 500 mg/100 ml or lower, particularly preferably 250 mg/100 ml or lower, irrespective of how the minimum content is defined. The minimum magnesium content is, for example, 1 μg/100 ml or higher, preferably 1 mg/100 ml or higher, more preferably 10 mg/100 ml or higher, further preferably 15 mg/100 ml or higher, still further preferably 20 mg/100 ml or higher, particularly preferably 35 mg/100 ml or higher, irrespective of the calcium content. The maximum magnesium content is, for example, 500 mg/100 ml or lower, preferably 350 mg/100 ml or lower, more preferably 100 mg/100 ml or lower, further preferably 75 mg/100 ml or lower, particularly preferably 50 mg/100 ml or lower, irrespective of how the minimum content is defined.

The liquid food composition of the present invention contains a vegetable protein. The vegetable protein is not particularly limited so long as a vegetable protein showing “a relative mobility (Rf value) at a cumulative value of pixel intensity frequency of 50%” obtained by densitometry analysis of SDS-PAGE pattern larger than 0.6 is used, but a protein showing the Rf value of 0.7 or larger, more preferably 0.8 or larger, is preferred. As for the maximum value of the “relative mobility (Rf value) at a cumulative value of pixel intensity frequency of 50%”, it is preferably 0.99 or smaller, more preferably 0.95 or smaller, further preferably 0.90 or smaller, irrespective of how the minimum value is defined. When the relative mobility (Rf value) is 0.6 or smaller, the vegetable protein is not sufficiently decomposed, and therefore the characteristic of the liquid food composition of the change of the form under the acidic condition may not be fully exhibited. Further, if the relative mobility (Rf value) is larger than 0.99, osmotic pressure of the composition becomes high, and comes to easily induce diarrhea.

The “pixel intensity” referred to in the present invention means a density value that can be obtained when an electrophoretic pattern is captured by a densitometer (optical density measurement apparatus), and it correlates with protein concentration. The SDS-PAGE is a method for separating constitutional molecules of proteins according to the molecular weights.

The “relative mobility” (Rf value, relative to front value) referred to in the present invention means a relative migration distance of each band in electrophoresis based on the migration distance of a preceding dye (bromophenol blue) observed in the electrophoresis, which is taken as “1”, and a value closer to “1” means a smaller molecular weight.

The “cumulative value of pixel intensity frequency of 50%” referred to in the present invention means “median of distribution” of constitutional molecules of vegetable protein separated by electrophoresis. Therefore, the “Rf value at the cumulative value of pixel intensity frequency of 50%” means the relative mobility corresponding to the “median of distribution” of the constitutional molecules of the vegetable protein, and an Rf value at the cumulative value of pixel intensity frequency of 50% closer to “1” means that the vegetable protein is more decomposed.

In the present invention, a lower content of globulins contained in the vegetable protein is preferred, and in particular, lower contents of 7S globulin and/or 11S globulin are more preferred. The contents of 7S globulin and 11S globulin in the vegetable protein and in the liquid food composition can be measured by densitometry analysis of an SDS-PAGE pattern.

Specifically, as for the minimum content of the 7S globulin, the content is preferably 0.01% by weight or higher, more preferably 0.05% by weight or higher, further preferably 0.1% by weight or higher. As for the maximum content of the 7S globulin, the content is preferably lower than 21% by weight, more preferably 15% by weight or lower, further preferably 6% by weight or lower, irrespective of how the minimum content is defined. If the 7S globulin content in the vegetable protein is lower than 0.01% by weight, osmotic pressure of the composition becomes high, so that it comes to easily induce diarrhea, and therefore such a content is not preferred. Further, if the 7S globulin content is 21% by weight or higher, decomposition of the vegetable protein becomes insufficient, so that the property of the liquid food composition to change the form thereof under the acidic condition may not be fully exhibited, and therefore such a content is not preferred. The 7S globulin content shall be calculated in terms of % by weight based on the vegetable protein.

As for the minimum content of the 11S globulin, the content is preferably 0.01% by weight or higher, more preferably 0.05% by weight or higher, further preferably 0.1% by weight or higher, based on the vegetable protein. As for the maximum content of the 11S globulin, the content is preferably lower than 41% by weight, more preferably 32% by weight or lower, further preferably 20% by weight or lower, irrespective of how the minimum content is defined. If the 11S globulin content in the vegetable protein is lower than 0.01% by weight, osmotic pressure of the composition becomes high, so that it comes to easily induce diarrhea, and therefore such a content is not preferred. Further, if the 11S globulin content is 41% by weight or higher, decomposition of the vegetable protein becomes insufficient, so that the property of the liquid food composition to change the form thereof under the acidic condition may not be fully exhibited, and therefore such a content is not preferred. The 11S globulin content shall be calculated in terms of % by weight based on the vegetable protein.

The contents of the 7S globulin and/or 11S globulin in the liquid food composition can be appropriately adjusted by controlling the amount of the vegetable protein to be added to the liquid food composition, and is not particularly limited. As for the minimum content of the 7S globulin based on the liquid food composition, specifically, the content is preferably 0.000025% by weight or higher, more preferably 0.001% by weight or higher, further preferably 0.01% by weight or higher, still further preferably 0.015% by weight or higher, particularly preferably 0.06% by weight or higher. As for the maximum 7S globulin content based on the liquid food composition, the content is preferably 4.2% by weight or lower, more preferably 3.0% by weight or lower, further preferably 2.1% by weight or lower, still further preferably 1.2% by weight or lower, particularly preferably 0.6% by weight or lower, irrespective of how the minimum content is defined. If the 7S globulin content based on the liquid food composition is lower than 0.000025% by weight, osmotic pressure of the composition becomes high, so that it comes to easily induce diarrhea, and therefore such a content is not preferred. Further, if the 7S globulin content based on the liquid food composition exceeds 4.2% by weight, decomposition of the vegetable protein becomes insufficient, so that the property of the liquid food composition to change the form thereof under the acidic condition may not be fully exhibited, and therefore such a content is not preferred.

As for the minimum content of the 11S globulin based on the liquid food composition, the content is preferably 0.000025% by weight or higher, more preferably 0.001% by weight or higher, further preferably 0.01% by weight or higher, further preferably 0.05% by weight or higher, particularly preferably 0.2% by weight or higher. As for the maximum content of the 11S globulin based on the liquid food composition, the content is preferably 8.2% by weight or lower, more preferably 6.4% by weight or lower, further preferably 4.0% by weight or lower, still further preferably 3.0% by weight or lower, particularly preferably 2.0% by weight or lower, irrespective of how the minimum content is defined. If the 11S globulin content based on the liquid food composition is lower than 0.000025% by weight, osmotic pressure of the composition becomes high, so that it comes to easily induce diarrhea, and therefore such a content is not preferred. Further, if the 11S globulin content exceeds 8.2% by weight, decomposition of the vegetable protein becomes insufficient, so that the property of the liquid food composition to change the form thereof under the acidic condition may not be fully exhibited, and therefore such a content is not preferred.

In the present invention, the vegetable protein can be selected from the group consisting of those of soybean, green peas, rice, wheat, corn, and so forth, and a vegetable protein derived from soybean is preferred in view of nutrition. When the vegetable protein originates in soybean, the 7S globulin is referred to as β-conglycinin, and the 11S globulin is referred as glycinin.

In the present invention, the vegetable protein is preferably a vegetable protein subjected to a decomposition treatment. The method for obtaining such a vegetable protein decomposed to a specific degree as mentioned above is not particularly limited, and any method may be used. Examples include, for example, the acidolysis method using an acid such as hydrochloric acid, the enzymatic hydrolysis method using an enzyme such as protease, and so forth. The conditions for performing these methods are not particularly limited, so long as a vegetable protein decomposed to the aforementioned degree can be obtained. In the present invention, the vegetable protein may be a vegetable protein of such a decomposition degree as mentioned above, and may not necessarily be a vegetable protein obtained by a decomposition treatment. As for soybean, for example, there are known a variety of soybean deficient in the 7S globulin molecule, and a variety of soybean deficient in the 11S globulin molecule (for example, Higashiyama No. 205), and the effect of the present invention can also be obtained by using a vegetable protein obtained from such varieties.

In the vegetable protein used in the present invention, the ratio of contents of the 11S globulin and 7S globulin [(11S globulin)/(7S globulin), weight basis] is preferably larger than 0.0005, more preferably 0.0007 or larger, further preferably 0.002 or larger, still further preferably 1 or larger, particularly preferably 2 or larger. The ratio of the 11S globulin and 7S globulin contents is preferably smaller than 4100, more preferably 800 or smaller, further preferably 400 or smaller, still further preferably 6.8 or smaller, particularly preferably 5 or smaller. If the ratio of the 11S globulin and 7S globulin contents is not larger than 0.0005, or not smaller than 4100, the solidification ratio under the acidic condition may decease, and therefore such a ratio is not preferred.

The ratio of contents of the 11S globulin and 7S globulin [(11S globulin)/(7S globulin), weight basis] can also be calculated from the contents of the 11S globulin and 7S globulin based on the liquid food composition. Specifically, this ratio is preferably larger than 0.0005, more preferably 0.0007 or larger, further preferably 0.002 or larger, still further preferably 1 or larger, particularly preferably 2 or larger. Further, the ratio of the 11S globulin and 7S globulin contents is preferably smaller than 4100, more preferably 800 or smaller, further preferably 400 or smaller, still further preferably 6.8 or smaller, particularly preferably 5 or smaller, irrespective of the other conditions. If the ratio of 11S globulin and 7S globulin contents is not larger than 0.0005, or not smaller than 4100, the solidification ratio under the acidic condition may decrease, and therefore such a ratio is not preferred.

In the present invention, the content of the vegetable protein is not particularly limited so long as it is used in an amount nutritionally sufficient for a person who ingests or gives the liquid food composition. However, the content is preferably 0.25% by weight or higher, more preferably 0.5% by weight or higher, further preferably 1.0% by weight or higher, still further preferably 2.0% by weight or higher, particularly preferably 4.0% by weight or higher, based on the liquid food composition. As for the maximum content of the vegetable protein, the content is preferably 20.0% by weight or lower, more preferably 10.0% by weight or lower, further preferably 7.5% by weight or lower, still further preferably 5.0% by weight or lower, based on the liquid food composition, irrespective of how the minimum content is defined. A content of the vegetable protein smaller than 0.25% by weight is not preferred in view of supply of the protein components. Further, a content larger than 20.0% weight poses a problem of increase in viscosity of the liquid food composition, and therefore such a content is not preferred.

In the present invention, the ratio of contents of the one or more kinds of substances selected from the group consisting of alginic acid, a salt thereof, and pectin, and the vegetable protein [(one or more kinds of substances selected from the group consisting of alginic acid, a salt thereof, and pectin)/(vegetable protein), weight basis] affects efficiency of the solidification of the liquid food composition under the acidic condition. Therefore, as for the minimum value of the content ratio, the ratio is preferably 0.05 or larger, more preferably 0.1 or larger, further preferably 0.2 or larger. As for the maximum value of the content ratio, the ratio is preferably 4.0 or smaller, more preferably 3.0 or smaller, further preferably 2.5 or smaller, still further preferably 2.0 or smaller, particularly preferably 1.0 or smaller. If the content ratio of the one or more kinds of substances selected from the group consisting of alginic acid, a salt thereof, and pectin, and the vegetable protein is smaller than 0.05, or larger than 4.0, the solidification ratio under the acidic condition may decrease, and therefore such a ratio is not preferred.

Further, in the present invention, the ratio of the contents of the one or more kinds of substances selected from the group consisting of alginic acid, a salt thereof, and pectin, and 7S globulin [(one or more kinds of substances selected from the group consisting of alginic acid, a salt thereof; and pectin)/(7S globulin), weight basis] affects the solidification of the liquid food composition under the acidic condition. Therefore, as for the minimum value of the content ratio, it is preferably larger than 0.07, more preferably 0.24 or larger, further preferably 0.5 or larger, still further preferably 0.8 or larger, particularly preferably 1.7 or larger. As for the maximum value of the ratio of the contents of the one or more kinds of substances selected from the group consisting of alginic acid, a salt thereof, and pectin, and 7S globulin, the ratio is preferably 200000 or smaller, more preferably 1000 or smaller, further preferably 100 or smaller, still further preferably 67 or smaller, particularly preferably 17 or smaller, irrespective of how the minimum ratio is defined. If the ratio of the contents of the one or more kinds of substances selected from the group consisting of alginic acid, a salt thereof, and pectin, and 7S globulin is not larger than 0.07, or larger than 200000, the solidification ratio under the acidic condition may decrease, and therefore such a ratio is not preferred. The ratio of the contents is calculated from the content of the one or more kinds of substances selected from the group consisting of alginic acid, a salt thereof, and pectin based on the liquid food composition, and the content of 7S globulin based on the liquid food composition.

Further, in the present invention, the ratio of the contents of the one or more kinds of substances selected from the group consisting of alginic acid, a salt thereof, and pectin, and 11S globulin [(one or more kinds of substances selected from the group consisting of alginic acid, a salt thereof, and pectin)/(11S globulin), weight basis] affects the solidification of the liquid food composition under the acidic condition. Therefore, as for the minimum value of the content ratio, the ratio is preferably larger than 0.04, more preferably larger than 0.12, further preferably 0.16 or larger, still further preferably 0.25 or larger, particularly preferably 0.5 or larger. As for the maximum value of the ratio of the contents of the one or more kinds of substances selected from the group consisting of alginic acid, a salt thereof, and pectin, and 11S globulin, the ratio is preferably 200000 or smaller, more preferably 1000 or smaller, further preferably 100 or smaller, still further preferably 20 or smaller, particularly preferably 5 or smaller, irrespective of how the minimum ratio is defined. If the ratio of the contents of the one or more kinds of substances selected from the group consisting of alginic acid, a salt thereof, and pectin, and 11S globulin is not larger than 0.04, or larger than 200000, the solidification ratio under the acidic condition may decrease, and therefore such a ratio is not preferred. The ratio of the contents is calculated from the content of the one or more kinds of substances selected from the group consisting of alginic acid, a salt thereof, and pectin based on the liquid food composition, and the content of 11S globulin based on the liquid food composition.

The liquid food composition of the present invention may also contain oil. Type of the oil is not particularly limited, and oil in the form of liquid, semi-solid, or solid may be used, or oil containing an aliphatic acid may be used. Specifically, the composition may contain such oils as soybean oil, corn oil, rapeseed oil, palm oil, palm seed oil, safflower oil, olive oil, perilla oil, sesame oil, fish oil, beef tallow, and lard, as well as, for example, oils containing a saturated fatty acid such as stearic acid, oils containing an unsaturated fatty acid such as oleic acid, α-linolenic acid, γ-linolenic acid, linoleic acid, eicosapentaenoic acid, docosahexaenoic acid, and arachidonic acid, oils containing a medium chain fatty acid (MCT) etc., and so forth, and these oils may also be used in combination. Further, the content of the oil in the liquid food composition is not particularly limited, and it may be an amount nutritionally sufficient for a person who ingests or gives the liquid food composition, although appropriate amount changes depending on the composition of the liquid food composition. Specifically, as for the minimum content of oil, it is, for example, 0.01% by weight or higher, preferably 0.20% by weight or higher, more preferably 0.50% by weight or higher, further preferably 1.00% by weight or higher, still further preferably 2.00% by weight or higher, particularly preferably 3.00% by weight or higher, more particularly preferably 3.40% by weight or higher. As for the maximum content of the oil, the oil is preferably contained at a content of 10.0% by weight or lower, more preferably 7.5% by weight or lower, still more preferably 5.0% by weight or lower, further preferably 4.0% by weight or lower, irrespective of how the minimum content is defined. A content of oil lower than 0.01% by weight is not preferred in view of supply of lipid components. If the content of oil is higher than 10.0% by weight, lipid components may be excessively ingested, and therefore such a content is not preferred.

The liquid food composition of the present invention may also contain an emulsifier. Type of the emulsifier is not particularly limited, and examples include, for example, lecithin, polyglycerol fatty acid esters, sorbitan fatty acid esters, propylene glycol fatty acid esters, stearoyl calcium lactate, lysolecithin, sucrose fatty acid esters, and so forth. Among these, lysolecithin and sucrose fatty acid esters are preferably used from the viewpoint of stabilization of emulsification of the liquid food composition. These emulsifiers may be used independently, or as a combination of two or more kinds of them, and the content of the emulsifier may be appropriately changed depending on the composition of the liquid food composition. Specifically, as for the minimum content of the emulsifier in the liquid food composition, the content is preferably higher than 0.17% by weight, more preferably 0.24% by weight or higher, further preferably 0.34% by weight or higher, irrespective of how the maximum content is defined. As for the maximum content of the emulsifier, the content is preferably 2.00% by weight or lower, more preferably 1.02% by weight or lower, further preferably 0.85% by weight or lower, still further preferably 0.68% by weight or lower, particularly preferably 0.51% by weight or lower. If the content of the emulsifier is 0.17% by weight or lower, stability of emulsification of the liquid food composition may be reduced, and therefore such a content is not preferred. If the content of the emulsifier is higher than 2.00% by weight, it results in increase in viscosity of the liquid food composition, and therefore such a content is not preferred. The content of the emulsifier may be adjusted as an amount relative to the amount of oil contained in the composition. Specifically, the content of the emulsifier is preferably 5% by weight or higher, more preferably 7% by weight or higher, further preferably 10% by weight or higher, based on the oil. Further, irrespective of how the minimum content is defined, the content is preferably 30% by weight or lower, more preferably 20% by weight or lower, further preferably 15% by weight or lower, based on the oil. If the content of the emulsifier with respect to the oil is lower than 5% by weight, the emulsification stability of the liquid food composition may be degraded, and therefore such a content is not preferred. Further, if the content of the emulsifier with respect to the oil is higher than 30% by weight, it results in increase in viscosity of the liquid food composition, and therefore such a content is not preferred.

The liquid food composition of the present invention may further contain food additives usable for common foods such as ordinary foods used as food additives, existing food additives, and natural flavoring agents, nutrient ingredients such as vitamins and minerals, ingredient derived from plants, mushrooms, animals, or microorganisms added to improve mouthfeel, taste, flavor, color, preservation property, quality, etc., for example, polysaccharide thickeners, dietary fibers, antifreezing materials, ice recrystallization inhibiting materials, and so forth, and these can be used in combination.

Since the liquid food composition of the present invention is a composition showing superior solidification ratio under the acidic condition, it provides reduced volume of liquid remaining after the change of the form in the stomach etc. (unsolidified portion). Therefore, it can be used for nutritious foods, enteral nutritious foods, thick liquid diets, diet foods, diets for patients with diabetes, kidney disease, etc., enteral nutrient preparations including those classified into drugs, and so forth, utilizing such an advantage as mentioned above. The liquid food composition of the present invention can be ingested orally, by tube feeding, or the like, and ingestion method therefor is not particularly limited. However, it is preferably used as a thick liquid diet, or an enteral nutritious food or enteral nutritional agent, which is ingested by tube feeding via a nasogastric tube, gastrostomy tube, or the like.

The liquid food composition of the present invention can be a liquid food composition contained in a container, in which a predetermined volume (for example, not smaller than 100 ml and not larger than 500 ml) of the composition is filled in a container connectable with a nasogastric catheter or gastrostomy catheter.

The liquid food composition of the present invention can be used for elderly people, patients with diseases, preoperative or postoperative patients, or healthy subjects. It is particularly preferably used for those who have a disease or pathological condition, and require liquid foods (medical foods) or tube feeding. Examples of such disease or condition include insufficient biting power or swallowing power due to advanced age, reduction of deglutition power or dysphagia (late effect of cerebral stroke, amyotrophic lateral sclerosis, etc.), asitia due to central nerve disease (dementia disease etc.), asitia due to cancerous cachexia etc. (terminal cancer case etc.), stenosis from pharynx to cardia (pharyngeal cancer, esophageal cancer, gastric cardia cancer, etc.), disease for which elemental diet therapy is effective (Crohn's disease etc.), and gastroesophageal reflux diseases (including non-erosive gastroesophageal reflux disease, reflux esophagitis, and barrett esophagi).

In the present invention, the expression “A and/or B” is used to mean both A and B, or either one of A and B, unless especially indicated.

### EXAMPLES

Hereafter, the present invention will be specifically explained with reference to the following examples and comparative example. However, the present invention is not limited by them.

### <Test for Confiuning Solidification Ratio Under Acidic Condition>

(1) Artificial gastric juice (Japanese Pharmacopoeia, Disintegration test, 1st fluid, precisely, a solution prepared by dissolving 2.0 g of sodium chloride in 7.0 mL of hydrochloric acid and water to make 1000 mL, this solution is clear and colorless, and has a pH of about 1.2) kept at 37° C. in an amount of 20 g (used as [weight of artificial gastric juice]) was added to a 50 ml volume plastic tube.
(2) The liquid food composition (10 g, 25° C.) was added to the artificial gastric juice, and weight of the plastic tube containing the artificial gastric juice and the liquid food composition was measured (this is used as [weight of tube before filtration]).
(3) The content in the plastic tube was gently stirred by using “HL-2000 HybriLinker” (UVP Laboratory Products). More precisely, the tube was fixed with the immobilization device in the chamber, the motor control mode was adjusted to the minimum level by switching the Motor Control dial of the apparatus to the indication of “MIN”, and then the content was stirred under the conditions of 37° C. for 2 minutes and 30 seconds.
(4) The solid matter was subjected to suction filtration on a nylon screen (40 mesh, SOGO LABORATORY GLASS WORKS), of which weight was measured in advance, to remove the liquid portion, the residue was placed on paper towel or the like together with the nylon screen to remove excessive moisture over 2 minutes, and weight of the solid matter including the nylon screen was measured (it is used as [weight of solid matter after filtration]). Further, after the content liquid in the tube was emptied, moisture remaining in the tube was removed, and weight of the plastic tube was measured (it is used as [Weight after filtration]).
(5) The solid matter remained on the nylon screen was confirmed, and the solidification ratio was calculated in accordance with the equation (1).

[Equation 1]

Solidification ratio=(Weight of solid matter after filtration)−(Weight of nylon screen)/(Weight of tube before filtration)−(Weight after filtration)−(Weight of artificial gastric juice)×100 Equation (1)

### <Methods for Calculating Cumulative Value of Pixel Intensity Frequency and Relative Mobility Corresponding to Cumulative Value of 50%>

### (1) Preparation of Sample for SDS-PAGE

A vegetable protein (4.4 g) was added to warm water (100 ml) at 50° C., and the mixture was stirred for 30 minutes to prepare a protein dispersion. Then, the protein dispersion diluted 5 times with distilled water (50 μl) and a buffer for SDS-PAGE sample of 2× concentration (50 μl) were mixed, and the mixture was heated with a block heater at 95° C. for 3 minutes to obtain a sample for SDS-PAGE.

### (2) Implementation of SDS-PAGE

SDS-PAGE was carried out by the “Laemmli method (Nature, 227, 680-685, 1970)”. “e-PAGEL:E-T/R/D 520L” (ATTO, gradient gel of 5 to 20% concentration) was used as the electrophoresis gel, “Precision PLUS Protein Standard” (Bio-Rad) was used as the protein molecular weight marker, and the other reagents were similar to those used in the Laemmli method. The sample for electrophoresis prepared in (1) mentioned above (7.5 μl) was applied to the well of the gel for electrophoresis, and then SDS-PAGE was carried out at a constant current of 20 mA/gel. After completion of the electrophoresis, the protein was stained with “Bio-Safe Coomassie Stain” (Bio-Rad).

### (3) Calculation of Cumulative Value of Pixel Intensity Frequency and Rf Value Corresponding to Cumulative Value of 50%

By using a densitometer (optical density meter), “Image Quant LAS4000” (CCD camera type image analysis device, GE Healthcare), the electrophoresis pattern was captured with a white transmission light (no filter, exposure time was 1/100 second), and densitometry data of the electrophoresis pattern were obtained with image analysis software, “Image Quant TL” (GE Healthcare).

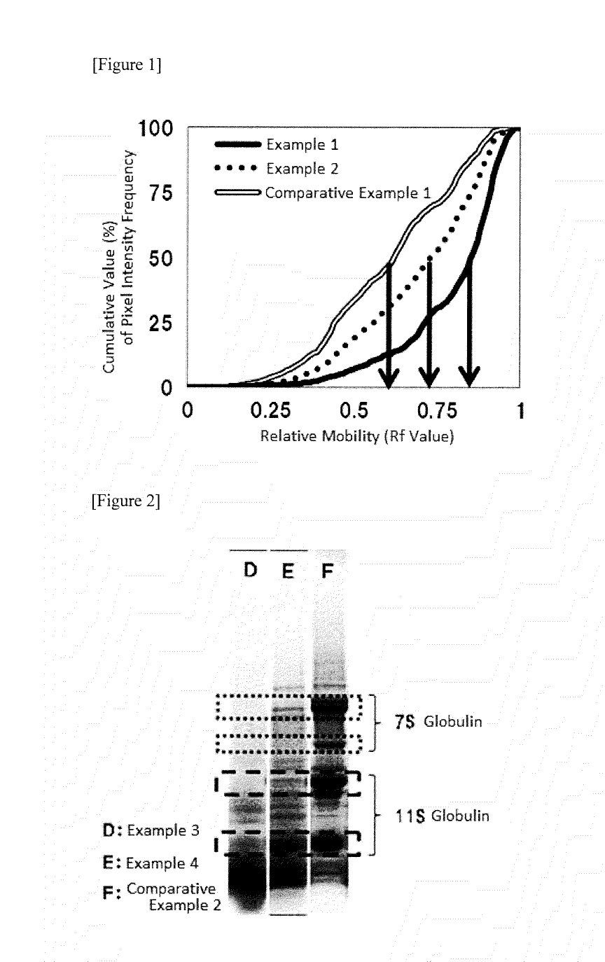

By using the densitometry data, “pixel intensity frequency (%)” was calculated in accordance with the equation: (Pixel intensity)/(Sum of total pixel intensities)×100, and then densitometry analysis results were obtained as a curve shown with a horizontal axis indicating “Rf value”, and a vertical axis indicating “cumulative value of pixel intensity frequency (%)” (FIG. 1). Furthermore, from the densitometry analysis results, a relative mobility (Rf value) corresponding to the cumulative value of 50% (corresponding to the cumulative value of 50% on the vertical axis) was obtained.

### <Test for Confirming Viscosity>

Viscosity of the liquid food composition was measured with a “Brookfield” type viscometer (TOKIMEC). More precisely, a measurement sample was put into a glass container having an internal diameter of 60 mm, the measurement was performed 3 times with the conditions of a liquid temperature of 25° C., rotor No. 2, revolution number of 60 rpm, and retention time of 30 seconds, and the average of the results was used as a measured value (viscosity).

### Example 1

A soybean protein raw material A was analyzed according to the description of <Methods for calculating cumulative value of pixel intensity frequency and relative mobility corresponding to cumulative value of 50%>, and densitometry analysis results were obtained as a curve shown with a horizontal axis indicating “Rf value” and a vertical axis indicating “cumulative value (%) of pixel intensity frequency” (FIG. 1, solid line). The soybean protein raw material A showed an Rf value of 0.8 as the Rf value corresponding to the cumulative value of 50% (corresponding to the cumulative value of 50% on the vertical axis) (Table 1).

Then, by using the soybean protein raw material A together with the other raw materials, of which composition is shown in Table 2, a liquid food composition was prepared. Viscosity of the liquid food composition was 170 cP (25° C.), and showed fluidity. Further, solidification ratio of the composition under the acidic condition was calculated according to the description of <Test for confirming solidification ratio under acidic condition>, and as a result, the composition showed a solidification ratio of 80%, and very favorably solidified (Table 3).

### Example 2

In the same manner as that described in Example 1, an Rf value corresponding to the cumulative value of 50% of a soybean protein raw material B was determined. The results of the densitometry analysis are shown in FIG. 1 with the dashed line. The Rf value corresponding to the cumulative value of 50% (corresponding to the cumulative value of 50% on the vertical axis) of the soybean protein raw material B was 0.7 (Table 1).

Then, by using the soybean protein raw material B together with the other raw materials, of which composition is shown in Table 2, a liquid food composition was prepared. After the preparation, the liquid food composition was evaluated in the same manner as that of Example 1. As a result, viscosity of the liquid food composition was 190 cP (25° C.), the solidification ratio under the acidic condition was 65%, and thus the composition favorably solidified (Table 3).

### Comparative Example 1

In the same manner as that described in Example 1, an Rf value corresponding to the cumulative value of 50% of a soybean protein raw material C was determined. The results of the densitometry analysis are shown in FIG. 1 with the double line. The Rf value corresponding to the cumulative value of 50% (corresponding to the cumulative value of 50% on the vertical axis) of the soybean protein raw material C was 0.6 (Table 1).

Then, by using the soybean protein raw material C together with the other raw materials, of which composition is shown in Table 2, a liquid food composition was prepared. After the preparation, the liquid food composition was evaluated in the same manner as that of Example 1. As a result, viscosity of the liquid food composition was 220 cP (25° C.), the solidification ratio under the acidic condition was 55%, and thus the composition solidified, but the solidification ratio was low (Table 3).

TABLE 1

| | | | Comparative |
|---|---|---|---|
| | Example 1 | Example 2 | Example 1 |
| Rf value corresponding to | 0.8 | 0.7 | 0.6 |
| cumulative value of 50% | | | |

TABLE 2
Composition (unit, g)

| Sodium alginate | 1.0 | |
|---|---|---|
| Calcium carbonate | 0.19 | |
| Magnesium carbonate | 0.14 | |
| Phosphoric acid salt | 0.6 | |
| Soybean protein | 4.4 | |
| Lysolecithin (emulsifier) | 0.34 | |
| Corn oil (lipid) | 3.4 | |
| Dextrin (saccharide) | 12 | |
| Vitamins, other minerals | 0.7 | |
| Distilled water | Reminder | |
| Total (volume) | 100 | ml |

TABLE 3

| | | | Comparative |
|---|---|---|---|
| | Example 1 | Example 2 | Example 1 |
| Viscosity (25° C.) | 170 cP | 100 cP | 220 cP |
| Solidification ratio | 80% | 65% | 55% |

### Example 3

### Method for Determining 7S Globulin and/or 11S Globulin Contents

### (1) Preparation of Sample for SDS-PAGE and Implementation of Electrophoresis

According to the descriptions of <Methods for calculating cumulative value of pixel intensity frequency and relative mobility corresponding to cumulative value of 50%>, (1) and (2) mentioned above, a sample for SDS-PAGE was prepared, and electrophoresis was performed.

### (2) Determination of 7S Globulin (Glycinin) and 11S Globulin (β-Conglycinin) Contents

By using a densitometer (optical density meter), “Image Quant LAS4000” (CCD camera type image analysis device, GE Healthcare), the electrophoresis pattern was captured with a white transmission light (no filter, exposure time was 1/100 second), and by using image analysis software, “Image Quant TL” (GE Healthcare), contents of 7S globulin (β-conglycinin) and 11S globulin (glycinin), which are constituent molecules of vegetable protein, were determined.

That is, after the electrophoresis pattern was captured with the CCD camera type image analysis device, densitogram data were obtained with the image analysis software. Then, by using the densitogram data, peak areas of the 11S globulin molecule and 7S globulin molecule, and peak areas of molecular weight markers of known concentrations were compared to determine the molecular weights. Further, the total amount of the proteins contained in the sample used for SDS-PAGE was measured (Micro BCA Protein Assay Kit, PIERCE), and contents of 7S globulin (β-conglycinin) and 11S globulin (glycinin) in the vegetable protein raw material were calculated.

According to the descriptions of <Method for determining 7S globulin and/or 11S globulin contents> mentioned above, SDS-PAGE pattern of a soybean protein raw material D was obtained (FIG. 2, D). Further, 11S globulin (glycinin), 7S globulin (β-conglycinin), and the total protein were quantified.

The 7S globulin (β-conglycinin) content was 6% by weight, and the 11S globulin (β-glycinin) content was 20% by weight, as calculated from the electrophoresis pattern of the soybean protein raw material D (Table 4). The Rf value corresponding to the cumulative value of 50% (corresponding to the cumulative value of 50% on the vertical axis) of the soybean protein raw material D was 0.8 or larger.

Then, by using the soybean protein raw material D together with the other raw materials, of which composition is shown in Table 2, a liquid food composition was prepared. Viscosity of the liquid food composition was 150 cP (25° C.), and showed fluidity under the neutral condition. Further, solidification ratio of the composition under the acidic condition was calculated according to the descriptions of <Test for confirming solidification ratio under acidic condition>, and as a result, the composition showed a solidification ratio of 79%, and extremely favorably solidified (Tables 5 and 6).

### Example 4

In the same manner as that described in Example 3, SDS-PAGE pattern of a soybean protein raw material E was obtained (FIG. 2, E), and then 11S globulin (glycinin), 7S globulin (β-conglycinin), and the total protein were quantified.

The 7S globulin (β-conglycinin) content was 15% by weight, and the 11S globulin (glycinin) content was 32%, as calculated from the electrophoresis pattern of the soybean protein raw material E (Table 4). The Rf value corresponding to the cumulative value of 50% (corresponding to the cumulative value of 50% on the vertical axis) of the soybean protein raw material E was 0.7 or larger.

Then, by using the soybean protein raw material E together with the other raw materials, of which composition is shown in Table 2, a liquid food composition was prepared. After the preparation, the liquid food composition was evaluated in the same manner as that of Example 1. As a result, viscosity of the liquid food composition was 180 cP (25° C.), and showed fluidity under the neutral condition. Further, solidification ratio of the composition under the acidic condition was calculated according to the descriptions of <Test for confirming solidification ratio under acidic condition>, and as a result, the composition showed a solidification ratio of 70%, and favorably solidified (Tables 4 and 5).

### Comparative Example 2

SDS-PAGE pattern of a soybean protein raw material F was obtained in the same manner as that described in Example 3 (FIG. 2, F), and then 11S globulin (glycinin), and 7S globulin (β-conglycinin) were quantified.

The 7S globulin (β-conglycinin) content was 21% by weight, and the 11S globulin (glycinin) content was 41%, as calculated from the electrophoresis pattern of the soybean protein raw material F (Table 4). The Rf value corresponding to the cumulative value of 50% (corresponding to the cumulative value of 50% on the vertical axis) of the soybean protein raw material F was 0.6 or smaller.

Then, by using the soybean protein raw material F together with the other raw materials, of which composition is shown in Table 2, a liquid food composition was prepared. After the preparation, the liquid food composition was evaluated in the same manner as that of Example 1. As a result, viscosity of the liquid food composition was 210 cP (25° C.), and showed fluidity under the neutral condition, but the fluidity was degraded. Further, solidification ratio of the composition under the acidic condition was calculated according to the descriptions of <Test for confirming solidification ratio under acidic condition>, and as a result, the composition showed a solidification ratio of 51%, and solidified, but the solidification ratio was low (Tables 4 and 5).

TABLE 4

| | | | Comparative |
|---|---|---|---|
| | Example 3 | Example 4 | Example 2 |
| Rf value corresponding to | 0.8 or | 0.7 or | 0.6 or |
| cumulative value of 50% | larger | larger | smaller |
| 7S globulin (μg) | 2 | 5 | 7 |
| 11S globulin (μg) | 7 | 11 | 14 |
| Total protein (μg) | 35 | 34 | 34 |
| 7S globulin content (% by weight, | 6 | 15 | 21 |
| based on proteins) | | | |
| 11S globulin content (% by weight, | 20 | 32 | 41 |
| based on proteins) | | | |
| Content ratio 11S/7S (based on | 3.3 | 2.1 | 2.0 |
| proteins, weight basis) | | | |

TABLE 5

| | | | Comparative |
|---|---|---|---|
| | Example 3 | Example 4 | Example 2 |
| Viscosity (25° C.) | 150 cP | 180 cP | 210 cP |
| Solidification ratio | 79% | 70% | 51% |

### Example 5

By using the soybean protein raw material D as the vegetable protein, liquid food compositions were produced with vegetable protein contents of 0.25% by weight (condition 1), 1% by weight (condition 2), 10% by weight (condition 3), and 20% by weight (condition 4). The raw materials other than the vegetable protein were added according to the composition shown in Table 2. The produced liquid food compositions were subjected to a homogenization treatment using a Manton Gaulin type high pressure homogenizer (Rannie 2000, APV) at 20 MPa (first time), and 48 MPa (second time), and then subjected to sterilization (F value, 8) in a retort sterilization machine. Solidification ratios of the compositions were calculated according to the descriptions of <Test for confirming solidification ratio under acidic condition>. As a result, the compositions produced with the conditions 1 to 4 showed solidification ratios of 58% or higher, and favorably solidified (Table 6).

### Example 6

By using an isolated soybean protein raw material G showing an Rf value of 0.8 corresponding to the cumulative value of 50% (corresponding to the cumulative value of 50% on the vertical axis), and having 7S globulin content in the protein raw material of 5.5% by weight, and 11S globulin content in the protein raw material of 20% by weight, together with the other raw materials, of which composition is shown in Table 2, a liquid food composition was produced. The produced liquid food compositions was further subjected to a homogenization treatment and sterilization treatment in the same manner as that described in Example 5, and solidification ratio was calculated according to the descriptions of <Test for confirming solidification ratio under acidic condition>. As a result, the solidification ratio was 81%, and the composition extremely favorably solidified (Table 6).

### Example 7

By using an isolated soybean protein raw material H showing an Rf value of 0.7 corresponding to the cumulative value of 50% (corresponding to the cumulative value of 50% on the vertical axis), and having 7S globulin content in the protein raw material of 14% by weight, and 11S globulin content in the protein raw material of 30% by weight, together with the other raw materials, of which composition is shown in Table 2, a liquid food composition was produced. The produced liquid food compositions was further subjected to a homogenization treatment and sterilization treatment in the same manner as that described in Example 5, and solidification ratio was calculated according to the descriptions of <Test for confirming solidification ratio under acidic condition>. As a result, the solidification ratio was 72%, and the composition favorably solidified (Table 6).

TABLE 6

| | | | | Comparative | Example 5 | | | | | |
|---|---|---|---|---|---|---|---|---|---|---|
| | | Example 3 | Example 4 | Example 2 | Condition 1 | Condition 2 | Condition 3 | Condition 4 | Example 6 | Example 7 |
| Vegetable protein raw | | Raw | Raw | Raw | Raw material D | | | | Raw | Raw |
| material | | material D | material E | material F | | | | | material G | material H |
| Rf value corresponding to | | 0.8 or | 0.7 or | 0.6 or | 0.8 or larger | | | | 0.8 | 0.7 |
| cumulative value of 50% | | larger | larger | smaller | | | | | | |
| Content | (1) Na | 0.23 | | 0.23 | 4.0 | 1.0 | 0.10 | 0.05 | 0.23 | |
| ratio | Alginate/vegetable | | | | | | | | | |
| | protein (based on | | | | | | | | | |
| | composition, weight | | | | | | | | | |
| | basis) | | | | | | | | | |
| | (2) 11S/7S (based | 3.2 | 2.1 | 2.0 | 3.3 | | | | 3.6 | 2.1 |
| | on composition, | | | | | | | | | |
| | weight basis) | | | | | | | | | |
| | (3) Na Alginate/7S | 3.8 | 1.5 | 1.1 | 67 | 17 | 1.7 | 0.80 | 4.4 | 1.6 |
| | (based on | | | | | | | | | |
| | composition, | | | | | | | | | |
| | weight basis) | | | | | | | | | |
| | (4) Na Alginate/11S | 1.1 | 0.71 | 0.55 | 20 | 5.0 | 0.50 | 0.25 | 1.1 | 0.76 |
| | (based on | | | | | | | | | |
| | composition, | | | | | | | | | |
| | weight basis) | | | | | | | | | |

| | | | | | | | | | |
|---|---|---|---|---|---|---|---|---|---|
| Solidification ratio | 79% | 70% | 51% | 58% | 60% | 80% | 85% | 81% | 72% |

1. A liquid food composition, comprising:
at least one substance selected from the group consisting of alginic acid, a salt thereof, and pectin;
a divalent metal salt; and
a vegetable protein having a relative mobility, Rf value, of larger than 0.6 at a cumulative value of pixel intensity frequency of 50% in SDS-PAGE densitometry analysis,
wherein the liquid food composition has fluidity at pH 5.5 to 10.0, and thickens and/or solidifies at a pH of lower than 5.5.

2. The liquid food composition according to claim 1, wherein the vegetable protein includes a 7S globulin at a content of not lower than 0.01% by weight and lower than 21% by weight, and the vegetable protein includes a 11S globulin at a content of not lower than 0.01% by weight and lower than 41% by weight.

3. The liquid food composition according to claim 2, wherein a content ratio of the 11S globulin with respect to the 7S globulin is larger than 0.0005 and smaller than 4100.

4. The liquid food composition according to claim 1, wherein a content ratio of the at least one substance with respect to the vegetable protein is from 0.05 to 4.0.

5. The liquid food composition according to claim 2, wherein a content ratio of the at least one substance with respect to the 7S globulin is larger than 0.07 and not larger than 200000.

6. The liquid food composition according to claim 2, wherein a content ratio of the at least one substance with respect to the 11S globulin is larger than 0.04 and not larger than 200000.

7. The liquid food composition according to claim 1, wherein the vegetable protein consists of at least one soybean protein.

8. The liquid food composition according to claim 2, wherein the 7S globulin is β-conglycinin.

9. The liquid food composition according to claim 2, wherein the 11S globulin is glycinin.

10. The liquid food composition according to claim 1, wherein the divalent metal salt comprises at least one of a calcium compound and a magnesium compound.

11. The method according to claim 12, wherein the feeding comprises feeding the liquid food composition filled in a container connectable with a nasogastric catheter or a gastrostomy catheter.

12. A method of treating a disease or a condition that requires liquid food or tube feeding, comprising:
feeding the liquid food composition according to claim 1 to a subject.

13. A method for improving thickening and/or solidification of a liquid food composition at a pH lower than 5.5, comprising:
preparing a liquid food composition including at least one substance selected from the group consisting of alginic acid, a salt thereof, and pectin, a divalent metal salt, and a vegetable protein,
wherein the vegetable protein has an Rf value of larger than 0.6.

14. The liquid food composition according to claim 2, wherein a content ratio of the at least one substance with respect to the vegetable protein is from 0.05 to 4.0.

15. The liquid food composition according to claim 3, wherein a content ratio of the at least one substance with respect to the vegetable protein is from 0.1 to 2.5.

16. The liquid food composition according to claim 3, wherein a content ratio of the at least one substance with respect to the 7S globulin is larger than 0.07 and not larger than 200000.

17. The liquid food composition according to claim 4, wherein a content ratio of the at least one substance with respect to the 7S globulin is from 1.7 to 17.

18. The liquid food composition according to claim 3, wherein a content ratio of the at least one substance with respect to the 11S globulin is larger than 0.04 and not larger than 200000.

19. The liquid food composition according to claim 4, wherein a content ratio of the at least one substance with respect to the 11S globulin is from 0.5 to 5.

20. The liquid food composition according to claim 1, wherein the divalent metal salt comprises a calcium compound and a magnesium compound.
